(19) United States
(12) Patent Application Publication
(10) Pub. No.: US 2009/0270762 A1
(43) Pub. Date: Oct. 29, 2009

(54) MEDICAL ADAPTER AND CHEMICAL REACTION CARTRIDGE

(75) Inventors: Takeo Tanaami, Musashino-shi (JP); Atsushi Ito, Musashino-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: YOKOGAWA ELECTRIC CORPORATION, Tokyo (JP)

(21) Appl. No.: 12/365,489

(22) Filed: Feb. 4, 2009

(30) Foreign Application Priority Data
Apr. 24, 2008 (JP) 2008-113395

(51) Int. Cl. A61B 5/157

(52) U.S. Cl. 600/576

(57) ABSTRACT

A medical adapter ensuring a safety and a chemical reaction cartridge adapted to the medical adapter are provided. Both ends of an injection needle are covered with a cover member. When the blood collection tube is inserted in the interior of the cover member via one opening section, one end of the injection needle smashes into a plug to be inserted in the blood collection tube, and the tip end of the injection needle reaches blood inside the blood collection tube. Further, when an insertion section of the cartridge is inserted in the interior of the cover member via the other opening section, the other end of the injection needle in inserted in the interior of the cartridge.

### FIELD OF THE INVENTION

The invention relates to a medical adapter for connecting a first object to be connected (hereinafter referred to as first connection object) and a second object to be connected (hereinafter referred to as second connection object) to each other so as to enable contents to flow between the first and second connection objects, and a chemical reaction cartridge connectable to the medial adapter.

### BACKGROUND OF THE INVENTION

There has been proposed, in Patent Document 1, a chemical reaction cartridge comprising an elastic body having chambers and flow paths formed therein and enabling fluid to flow in the chambers and paths or preventing fluid from flowing therein, and a substrate for retaining positions and shapes of the elastic body in order to particularly implement processing of synthesis, dissolution, detection, separation and so forth of a solution in accordance with a predetermined protocol with safety and ease without individual difference.

According to the invention disclosed in Patent Document 1, the elastic body is deformed by rollers and so forth to crash the chambers and paths, and in a state where the chambers and the paths are crashed, the rollers and so forth are moved or stopped, thereby enabling fluid to flow or preventing fluid from flowing.

The chemical reaction cartridge as disclosed in Patent Document 1 is also utilized as a biocartridge (biochip) for detecting/analyzing DNA, RNA, protein and so forth. In the paragraph [0033] and FIG. 21 of Patent Document 1, there is disclosed a biochip having a configuration wherein a recess is formed in an inlet of a sample such as blood and so forth, and an injection needle is taken out or put in the biochip via the recess so as to receive leaked sample in the recess.

Patent Document 2 discloses a biochip comprising a blood collection tube which is inserted in a syringe and transparent to an excited light or fluorescence to be excited, a rubber plug, a collection section in which collected blood is once held, a pre-processing section for implementing pre-processing such as separation, extraction, addition of fluorescent label, and so forth, and a substrate on which known DNA pieces are placed.

[Patent Document 1] JP2005-037368A

[Patent Document 2] JP No. 3757412 B2

According to the invention disclosed in Patent Document 1, there is less in possibility that leaked blood is attached to surroundings owing to the recess provided around the puncture site of the injection needle when the injection needle is taken out from the cartridge. However, there is a possibility that blood in the injection needle is attached to the surroundings when the injection needle is taken out or put in the biochip. Further, according to the invention disclosed in Patent Document 2, there is also a possibility that blood is attached to the surroundings when the blood collection needle is taken out or put in the rubber plug of the biochip.

### SUMMARY OF THE INVENTION

It is an object of the invention to provide a medical adapter (hereinafter referred to simply as an adapter) ensuring a safety and a chemical reaction cartridge (hereinafter referred to simply as cartridge) adapted to the medical adapter.

To achieve the above object, according to the invention, an adapter for connecting a first connection object and a second connecting to each other so as to enable contents to flow between the first and second connection objects, said adapter comprising a needle tube for contents through which the contents are caused to flow, a cover member fitted to the needle tube for contents and covering at least both ends of the needle tube for contents, wherein said cover member has a first receiving section for receiving the first connection object, and a second receiving section for receiving the second connection object, and wherein one end of the needle tube for contents is inserted in the first connection object when the first connection object is received in the first receiving section, and the other end of the needle tube for contents is inserted in the second connection object when the second connection object is received in the second receiving section.

According to the adapter, since both ends of the needle tube for contents are covered with the cover member while the first connection object is received in the first receiving section and the second connection object is received in the second receiving section, both the first connection object and second connection object are connected to each other, thereby ensuring a safety at the time of connecting operation.

The first connection object may be a cartridge capable of implementing chemical processing in accordance with a protocol defined by an internal structure thereof by causing the contents to flow by deformation generated upon application of an external force.

The adapter further comprise a needle tube for air for letting air out when the contents are caused to flow, wherein one end of the needle tube for air may be inserted in the first connection object received in the first receiving section or in the second connection object received in the second receiving section.

In a cartridge according to the invention capable of implementing chemical processing in accordance with a protocol defined by an internal structure thereof by causing the contents to flow by deformation generated upon application of an external force, said cartridge comprises a connection section connectable to an adapter for connecting the cartridge and other connection object to each other so as to enable the contents to flow between the cartridge and the other connection objects.

According to the cartridge, since there is provided the connection section connectable to the adapter for connecting the cartridge and the other connection object to each other, the adapter can be applied to the cartridge.

The adapter comprises a needle tube for contents for causing the contents to flow, a cover member fitted to the needle tube for contents and covering at least both ends of the needle tube for contents, wherein said cover member has a first receiving section for receiving the cartridge, and a second receiving section for receiving the other connection object, and wherein one end of the needle tube for contents is inserted in the cartridge via the connection section when the cartridge is received in the first receiving section, and the other end of the needle tube for contents is inserted in the other connection object when the other connection object is received in the second receiving section.

According to the adapter of the invention, since both ends of the needle tube for contents are covered with the cover member, and both the first connection object and second connection object are connected to each other when the first connection object is received in the first receiving section and the second connection object is received in the second receiving section, a safety at the time of connecting operation can be assured.

According to the cartridge of the invention, since there is provided the connection section connectable to the adapter for connecting the cartridge and the other connection object to each other, the adapter can be applied to the cartridge.

### BRIEF DESCRIPTION OF THE DRAWINGS

FIG. 1 is a front view showing a configuration of an adapter according to an embodiment of the invention;

FIGS. 2(A) to 2(C) are views showing usage of the adapter shown in FIG. 1, wherein FIG. 2(A) is view showing a state where a blood collection tube and a cartridge are inserted in the adapter, FIG. 2(B) is a view showing a positional relationship between the adapter, a blood collection tube and the cartridge, as viewed from above (along the line B-B of) FIG. 2(A), and FIG. 2(C) is a partly enlarged view of FIG. 2(B);

FIGS. 3(A) and 3(B) are views showing a configuration of an adapter according to another embodiment of the invention, wherein FIG. 3(A) is a front view showing an example of a cylindrically made-up adapter and FIG. 3(B) is a view showing a state where a blood collection tube and a cartridge are inserted in the adapter;

FIGS. 4(A) and 4(B) are views showing a configuration of an adapter according to still another embodiment of the invention, wherein FIG. 4(A) is a front view showing the adapter provided with a needle tube for air and FIG. 4(B) is a view showing a state where a blood collection tube and a cartridge are inserted in the adapter;

FIG. 5(A) is a front view showing a configuration of a linear adapter provided with a needle tube for air and FIG. 5(B) is a front view showing a configuration of a cartridge;

FIG. 6 is view showing a state where a blood collection tube and a cartridge are inserted in the adapter shown in FIG. 5(A); and

FIG. 7 is view showing an example of usage of an adapter as a connection adapter for connecting flow tubes to each other.

### BEST MODE FOR CARRYING OUT THE INVENTION

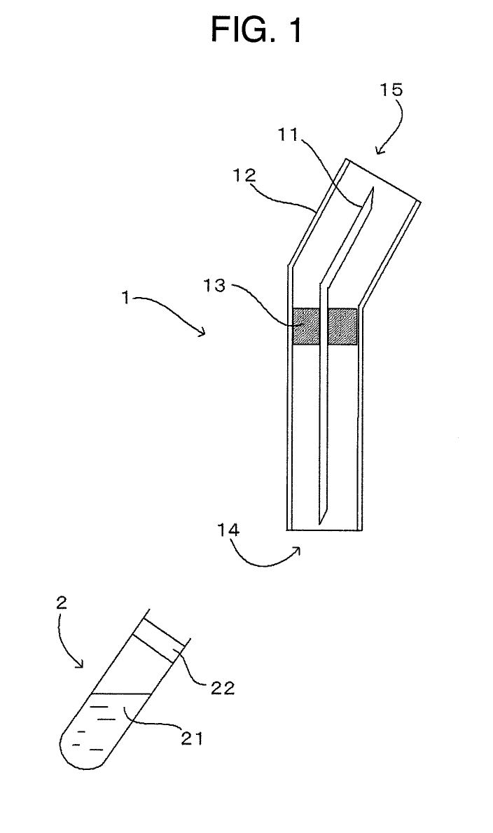

An adapter according to embodiments of the invention is now described with reference to FIGS. 1 to 7.

FIG. 1 is a front view showing a configuration of an adapter according to an embodiment of the invention.

As shown in FIG. 1, an adapter 1 according to an embodiment of the invention is provided with an injection needle 11 which is acute at both ends, a cover member for covering the injection needle 11, and a fixed member 13 for fixing the injection needle 11 to the interior of the cover member 12.

Further, as shown in FIG. 1, the injection needle 11 has a bent shape, and the cover member 12 is cylindrically formed along the shape of the injection needle 11 and has one opening section 14 at one end side and the other opening section 15 at the other end side. The cover member 12 extends outward beyond both ends of the injection needle 11 and envelops the entire injection needle 11 up to both ends thereof radially from all the direction.

A blood collection tube 2 shown in FIG. 1 is inserted in one opening section 14 of the cover member 12 and a cartridge 3, described later, is inserted in the other opening section 15 of the cover member 12.

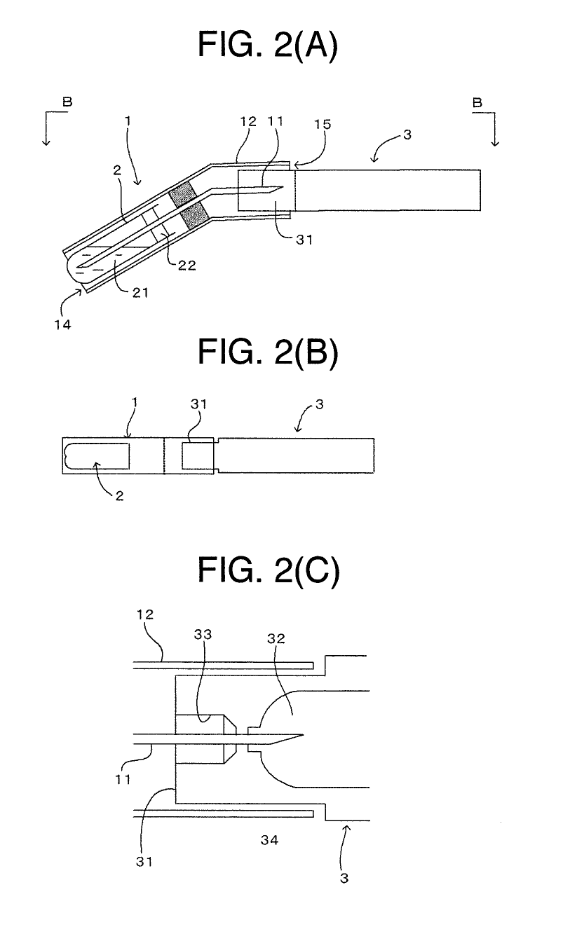

Usage of the adapter 1 is now described with reference to FIGS. 2(A) to 2(C).

FIG. 2(A) is view showing a state where the blood collection tube 2 and the cartridge 3 are inserted in the adapter 1.

As shown in FIG. 2(A), when the blood collection tube 2 is inserted in the interior of the cover member 12 via one opening section 14, one end of the injection needle 11 smashes into a plug 22 to be inserted in the blood collection tube 2, and the tip end of the injection needle 11 reaches blood 21 inside the blood collection tube 2. Further, when an insertion section 31 of the cartridge 3 is inserted in the interior of the cover member 12 via the other opening section 15, the other end of the injection needle 11 in inserted in the interior of the cartridge 3. At this point in time, since the adapter 1 has a bent shape, the cartridge 3 can be installed horizontally.

The cartridge 3 makes up a reactor which is generally called as a “microreactor”. The cartridge 3 is made up by a relatively hard substrate and a relatively soft elastic member having elasticity which is overlaid on the substrate. Glass, metal, resin and so forth are used as a material of the substrate and silicon rubber, PDMS (polydimethylsiloxane), natural rubber, polymer thereof, acrylic rubber, urethane rubber and so forth are used as a material of the elastic member.

The elastic member making up the cartridge 3 is provided with dents having shapes corresponding to chambers and flow paths, and chambers and flow paths are formed in the cartridge 3 between the elastic member and the substrate owing to the dents. With the configuration of the cartridge 3, it is possible to implement processing such as synthesis, dissolution, detection, separation and so forth of a solution in accordance with a predetermined protocol with safety and ease without individual difference. Meanwhile, the detail of the cartridge is disclosed in JP2005-037368A and so forth.

FIG. 2(B) is a view showing a positional relationship between the adapter 1, the blood collection tube 2 and the cartridge 3 as viewed from above (along the line B-B of) FIG. 2(A), and FIG. 2(C) is a partly enlarged view of FIG. 2(B).

As shown in FIG. 2(C), an insertion section 31 of the cartridge 3 has a recess 33 for receiving the injection needle 11. The recess 33 receives blood leaked from the cartridge 3 to prevent blood from diffusing outside.

As shown in FIGS. 2(B) and 2(C), when the insertion section 31 of the cartridge 3 is inserted in the interior of the cover member 12 via the other opening section 15, the tip end of the injection needle 11 is inserted in the cartridge 3 via the recess 33, and reaches a collection chamber 32 formed in the cartridge 3.

As mentioned above, one end of the injection needle 11 is inserted in blood 21 of the blood collection tube 2, and the other end of the injection needle 11 is inserted in the collection chamber 32, respectively, by merely inserting the blood collection tube 2 and the cartridge 3 in the adapter 1, respectively, so that the blood 21 can be safely introduced into the collection chamber 32 via the injection needle 11. Since the injection needle 11 is covered with the cover member 12 up to both ends thereof, it is possible to ensure a safety at the time of connecting operation.

Introduction of blood into the collection chamber 32 and an inspection of blood taken in the collection chamber 32 are implemented by operating the cartridge 3 in a state where the blood collection tube 2 and the adapter 1 are connected to each other. The inspection can be implemented in accordance with a predetermined protocol by moving blood and internal reagent in such a manner that the cartridge 3 is squeezed by use of a pressing means such as rollers, squeegees and so forth, as disclosed in JP2005-037368A and so forth.

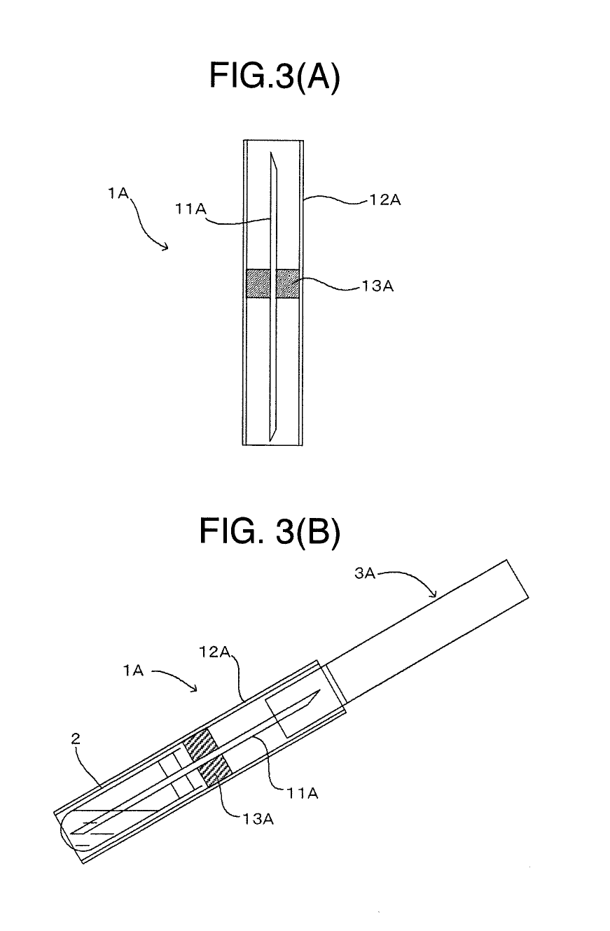

FIG. 3(A) is a front view showing an example of a cylindrically made-up adapter.

An adapter 1A shown in FIG. 3(A) is provided with an injection needle 11A which is acute at both ends, a cover member 12A for covering the injection needle 11A, and a fixed member 13A for fixing the injection needle 11A to the interior of the cover member 12A.

Further, as shown in FIG. 3(A), the injection needle 11A has a linear shape. The cover member 12A is cylindrically formed along the shape of the injection needle 11A and has opening sections formed respectively at both ends. The cover member 12A extends outward beyond both ends of the injection needle 11A and envelops the entire injection needle 11A up to both ends thereof radially from all the direction.

FIG. 3(B) is a view showing a state where a blood collection tube 2 and a cartridge 3A are inserted in the adapter 1A.

As shown in FIG. 3(B), when the blood collection tube 2 is inserted in the interior of the cover member 12A, one end of the injection needle 11A is inserted in the blood collection tube 2, and the tip end of the injection needle 11A reaches blood 21 inside the blood collection tube 2. Further, when the cartridge 3A is inserted in the interior of the cover member 12A, the other end of the injection needle 11A is inserted in the interior of the cartridge 3A.

Introduction of blood into the cartridge 3A and an inspection of blood taken in the cartridge 3A are implemented by operating the cartridge 3A in a state where the blood collection tube 2 and the adapter 1 are connected to each other. The inspection can be implemented in accordance with a predetermined protocol by moving blood and internal reagent in such a manner that the cartridge 3A is squeezed by use of a pressing means such as rollers, squeegees and so forth, as disclosed in JP2005-037368A and so forth.

In such a manner, if it is not necessary to implement the testing in a state where the cartridge 3A is placed horizontally, a linear adapter 1A can be used. The direction of the blood collection tube and the cartridge when implementing the testing is optional, and an adapter having the configuration corresponding to the direction thereof can be used. For example, if the blood collection tube 2 may be positioned immediately under the cartridge 3A or it may be positioned reversely over the cartridge 3A by vertically inverting the blood collection tube 2.

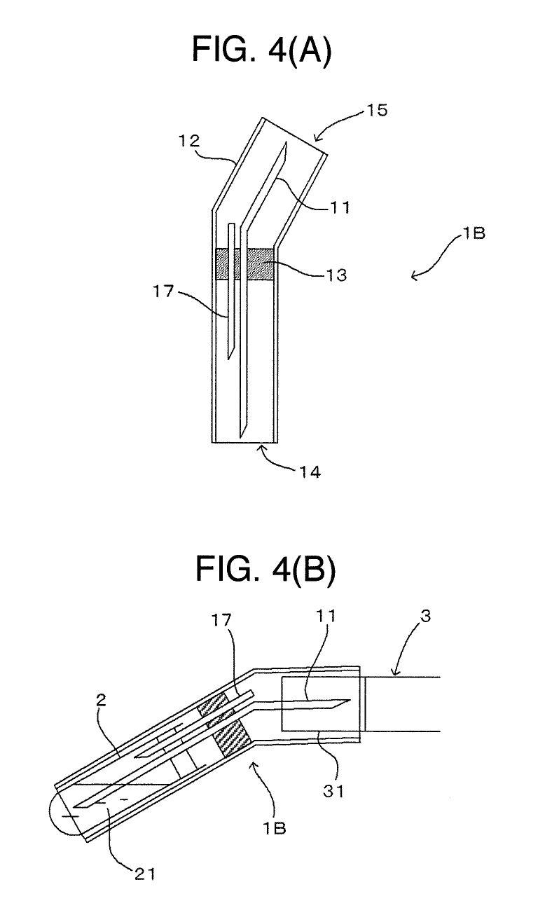

FIG. 4(A) is a front view showing an adapter provided with a needle tube for air.

As shown in FIG. 4(A), an adapter 1B according to an embodiment of the invention comprises an injection needle 11 which is acute at both ends, a needle tube 17 for air which is shorter than the injection needle 11 and has an acute one end, a cover member 12 for covering the injection needle 11, and a fixed member 13 for fixing the injection needle 11 and the needle tube 17 for air to the interior of the cover member 12.

Further, as shown in 4(A), the injection needle 11 has a bent shape, and the cover member 12 is cylindrically formed along the shape of the injection needle 11 and has one opening section 14 at one end side and the other opening section 15 at the other end side. The cover member 12 extends outward beyond respective both ends of the injection needle 11 and the needle tube 17 for air, and envelops the entire injection needle 11 and the entire needle tube 17 for air up to respective both ends thereof radially from all the direction.

Usage of the adapter 1B is described next with reference to FIG. 4(B).

FIG. 4(B) is a view showing a state where a blood collection tube 2 and a cartridge 3 are inserted in the adapter 1B.

As shown in FIG. 4(B), when the blood collection tube 2 is inserted in the interior of the cover member 12 via one opening section 14, one end of the injection needle 11 and one end of the needle tube 17 for air are inserted in the blood collection tube 2, and the tip end of the injection needle 11 reaches blood 21 inside the blood collection tube 2. Further, the tip end of the needle tube 17 for air is positioned in a space inside the blood collection tube 2.

Further, when an insertion section 31 of the cartridge 3 is inserted in the interior of the cover member 12 via the other opening section 15, the other end of the injection needle 11 is inserted in the interior of the cartridge 3. At this point in time, since the adapter 1B has a bent-shape, the cartridge 3 can be positioned horizontally.

Introduction of blood into the cartridge 3 and an inspection of blood taken in the cartridge 3 are implemented by operating the cartridge 3 in a state where the blood collection tube 2 and the adapter 1B are connected to each other. The inspection can be implemented in accordance with a predetermined protocol by moving blood and internal reagent in such a manner that the cartridge 3 is squeezed by use of a pressing means such as rollers, squeegees and so forth, as disclosed in JP2005-037368A and so forth.

In the case of using the adapter 1B, when blood 21 of the blood collection tube 2 is discharged from the injection needle 11, to be sucked into the cartridge 3, air is introduced into the blood 21 by the needle tube 17 for air. Accordingly, if the adapter 1B is used, it can cope with a case where there is no other path for introducing air into the blood collection tube 2.

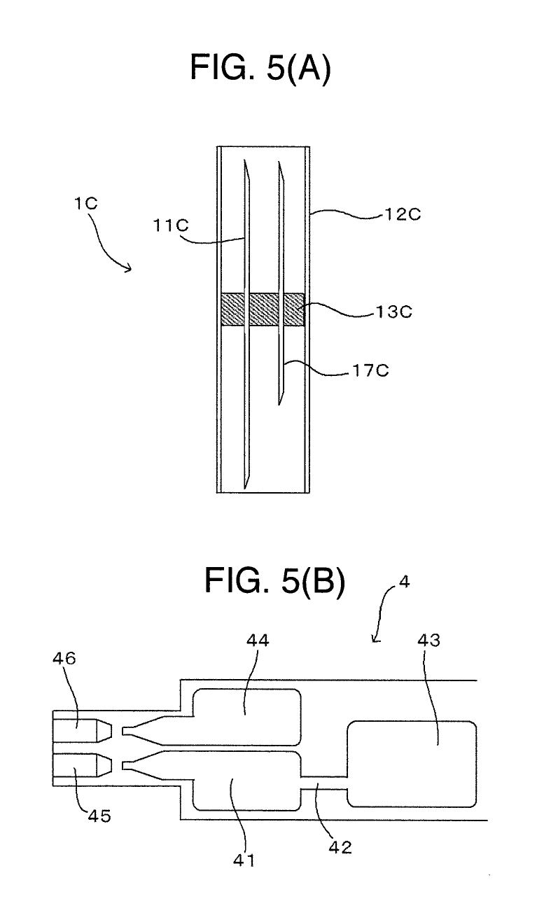

FIG. 5(A) is a front view of a linear adapter provided with a needle tube for air and FIG. 5(B) is a front view of a cartridge.

As shown in FIG. 5(B), an adapter 1C according to an embodiment of the invention comprises an injection needle 11C which is acute at both ends, a needle tube 17C for air which is shorter than the injection needle 11C and has acute both ends, a cover member 12C for covering the injection needle 11C and the needle tube 17C for air, and a fixed member 13C for fixing the injection needle 11C and needle tube 17C for air to the interior of the cover member 12.

Further, as shown in 5(A), the injection needle 11C and the needle tube 17C for air are linearly shaped. The cover member 12C is cylindrically formed along the shapes of the injection needle 11C and needle tube 17C for air and has opening sections at both ends. The cover member 12C extends outward beyond respective both ends of the injection needle 11C and needle tube 17C for air, and envelops the entire injection needle 11C and entire needle tube 17C up to respective both ends thereof radially from all the direction.

Meanwhile, as shown in FIG. 5(B), the cartridge 4 is provided with a blood collection tube 41 in which blood is injected, a chamber 43 connected to the blood collection tube 41 via a flow path 42, a discharge chamber 44 for discharging air, a recess 45 for receiving the injection needle 11C, a recess 46 for receiving needle tube 17C for air and other chambers and flow paths (not shown).

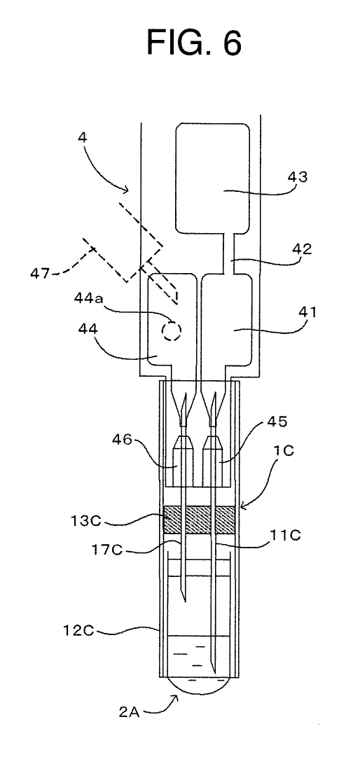

Usage of the adapter 1C and cartridge 4 is now described with reference to FIG. 6.

FIG. 6 is view showing a state where a blood collection tube 2A and a cartridge 4 are inserted in the adapter 1C.

As shown in FIG. 6, when the blood collection tube 2A is inserted in the interior of the cover member 12C, one end of the injection needle 11 and one of the of the needle tube 17C for air are inserted in the blood collection tube 2A, respectively, so that the tip end of the injection needle 11C reaches blood 21 inside the blood collection tube 2A. Further, the tip end of the needle tube 17C for air is positioned in a space inside the blood collection tube 2A.

Further, when the cartridge 4 is inserted in the interior of the cover member 12C, the other end of the injection needle 11C is inserted in the blood collection tube 41 via the recess 45, while the other end of the needle tube 17C for air is inserted in the discharge chamber 44 via the recess 46.

In this state, air in the discharge chamber 44 is pushed out via the needle tube 17C for air using rollers and so forth, thereby discharging air in the interior of the blood collection tube 2A. By so doing, an internal pressure of the blood collection tube 2A is increased so that blood is introduced into the blood collection tube 41 via the injection needle 11C. Further, as a different method, an air injection port 44a is provided in the discharge chamber 44, as shown in FIG. 6, so that air may be introduced into the discharge chamber 44 by a syringe, and so forth via the air injection port 44a. Still further, as shown in FIG. 6, a needle of a syringe 47 is pierced in the interior of the discharge chamber 44 from the outside of the cartridge 4 so that air may be introduced into the discharge chamber 44 by the syringe 47

An inspection of blood taken in the cartridge 4 is implemented by operating the cartridge 4 in a state where the blood collection tube 2A and adapter 1C are connected to each other. The inspection can be implemented in accordance with a predetermined protocol by moving blood and internal reagent in such a manner that the cartridge 3A is squeezed by use of a pressing means such as rollers, squeegees and so forth, as disclosed in JP2005-037368A and so forth.

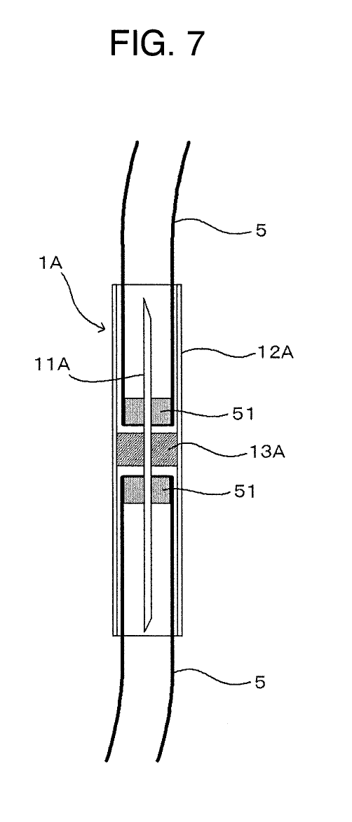

A scope of application of the adapter according to the invention is not limited. For example, as show in FIG. 7, the adapter 1A (shown in FIG. 3(A)) can be used as a connection adapter for connecting flow tubers 5, 5 to each other. In this case, when the flow tubes 5, 5 are inserted in both sides of the adapter 1A, the injection needle 11A smashes into plugs 51, 51 to be inserted in the interior of the flow tubes 5, 5 so that the flow tubes 5, 5 are connected to each other.

As mentioned in the foregoing, according to the adapter of the invention, both ends of the needle tube for contents are covered with the cove member, while the first connection object is received in the first receiving section and the second connection object is received in the second receiving section so that the first and second connection objects are connected to each other, thereby ensuring a safety at the time of connecting operation.

According to the cartridge of the invention, since there is provided the connection section connectable to the adapter for connecting the cartridge and the other connection object to each other, the adapter can be applied to the cartridge.

The scope of the invention is not limited to the foregoing embodiment. The invention can be widely applied to an adapter capable of ensuring a safety and a cartridge adapted to the adapter.

What is claimed is:

1. A medical adapter for connecting a first connection object and a second connecting to each other so as to enable contents to flow between the first and second connection objects, said medical adapter comprising:
a needle tube for contents through which the contents are caused to flow;
a cover member fitted to the needle tube for contents and covering at least both ends of the needle tube for contents;
wherein said cover member has a first receiving section for receiving the first connection object, and a second receiving section for receiving the second connection object, and
wherein one end of the needle tube for contents is inserted in the first connection object when the first connection object is received in the first receiving section, and the other end of the needle tube for contents is inserted in the second connection object when the second connection object is received in the second receiving section.

2. The medical adapter according to claim 1, wherein the first connection object is a chemical reaction cartridge capable of implementing chemical processing in accordance with a protocol defined by an internal structure thereof by causing the contents to flow by deformation generated upon application of an external force.

3. The medical adapter according to claim 1 further comprises a needle tube for air for letting air out when the contents are caused to flow;
wherein one end of the needle tube for air is inserted in the first connection object received in the first receiving section or in the second connection object received in the second receiving section.

4. A chemical reaction cartridge capable of implementing chemical processing in accordance with a protocol defined by an internal structure thereof by causing contents to flow by deformation generated upon application of an external force, said chemical reaction cartridge comprises:
a connection section connectable to a medical adapter for connecting the cartridge and other connection object to each other so as to enable contents to flow between the cartridge and the other connection objects.

5. The chemical reaction cartridge according to claim 4, wherein the medical adapter comprises:
a needle tube for contents through which the contents are caused to flow;
a cover member fitted to the needle tube for contents and covering at least both ends of the needle tube for contents;
wherein said cover member has a first receiving section for receiving the chemical reaction cartridge, and a second receiving section for receiving the other connection object, and
wherein one end of the needle tub for contents is inserted in the chemical reaction cartridge via the connection section when the chemical reaction cartridge is received in the first receiving section, and the other end of the needle tube for contents is inserted in the other connection object when the other connection object is received in the second receiving section.
